SEQUENCE LISTING (11 sequences)

SEQ ID NO: 1 - DNA, 848 bp, Myxine glutinosa
  CDS (4)..(543)
gtcatgaagtccttgtgtgtccccgcggttctctctctggtcctgatcctgctcttggatcaagcacctacagctcgggcagatgattctttgtccaaagagcaggtggagaatgcagttgatgaagcgttggacaagctgaacaaacagcaggtgtccacacgtaaactggcattgtctgaacaacaggatatccaggcagatgaaacagacgttgaaggacagttcaccatcaaatttgacgtggttgagaccgagtgcaatgcggacgatcccagggactgggccgattgtccgatcgcgacggactcgacaccggtcgatgcacagtgtgaggtcaccgtgttgagcaccgaggactccttggacgtcggagacgcgacttgcgatttcaacaggacggacggaaatgcgaggcgacgacgtggctggtttaagaaagcctggagaaaagtgaagcatgcgggacgacgagttcttgataccgcgaagggtgtgggaagacattatttgaataattggcttaatcgttatcgcggttaggaggaagctctgtgttggtggtggtggtggtggtggtggtggtggtgttgtgggttaatgggtgggagcgaatgatttctccccgacattcaatacttgctcgttaattccaattgattcgcttagttacacaagtaagccgaacaggtaaccagttagtaagctttaaaattgaacttttaggattgaaactgcagctgaactttttgtagtttaacatttaattgtgactctgctattaaactttgtctgatcatggaaataaaggattgcaaaaatgaaaaaaaaaaaaaaaaaaaaaaaaa

SEQ ID NO: 2 - protein, 179 aa, Myxine glutinosa
MKSLCVPAVLSLVLILLLDQAPTARADDSLSKEQVENAVDEALDKLNKQQVSTRKLALSEQQDIQADETDVEGQFTIKFDVVETECNADDPRDWADCPIATDSTPVDAQCEVTVLSTEDSLDVGDATCDFNRTDGNARRRRGWFKKAWRKVKHAGRRVLDTAKGVGRHYLNNWLNRYRG

SEQ ID NO: 3 - DNA, 863 bp, Myxine glutinosa
  CDS (2)..(508)
cttgtgtgtcccagcggttctctctctggtcctgatcctgctcttggatcaagcacctacagctcgggcagatgattctttgtccaacgagcaggtggagaatgcagttgatgaagcgttggacaagctgaacaatcagcaggtgtccacacgtaaactggcattgtctgaacaacaggatatccaggcagatgaaacagacgttgaaggacagaccaccatcatattttacgtggttgagaccgagtgcaatgcggacgatcccagggactgggccgattgtccgatcgcgacggactcgccaccgggcattgcacagtgtgaggtcaccgtgttgagcaccgaggactccttggacgtcggagacgcgacttgcgatttcaactcgacgggcggaaatgcgaggcgacgacgtggctggtttaagaaagcctggagaaaagtgaagaatgcgggacgagttcttaagggtgtgggaatacattatggtgttggattaatcggttaatcggttggattaatcgttatcacggttcggaggaagtcctctgttggtggtggtggtggtggtggtggtggtggtggtggtggtggtggtggtggtgtggggggttatgggttaatgggtgggagcgaatgatttctccccgacattcaatacttgctcgttaattccaattgattcgcttagttacacaagtaagccgaacaggtaaccagttagtaagctttaaaattgaacttttaggattgaaactgcagctgaactttttgtagtttaacatttaattgtgactctgctattaaactttgtctgatcatggaaataaaggattgcaaaaatgaaaaaaaaaaaaaaaaaa

SEQ ID NO: 4 - protein, 168 aa, Myxine glutinosa
LCVPAVLSLVLILLLDQAPTARADDSLSNEQVENAVDEALDKLNNQQVSTRKLALSEQQDIQADETDVEGQTTIIFYVVETECNADDPRDWADCPIATDSPPGIAQCEVTVLSTEDSLDVGDATCDFNSTGGNARRRRGWFKKAWRKVKNAGRVLKGVGIHYGVGLIG

SEQ ID NO: 5 - protein, 38 aa, Myxine glutinosa
GWFKKAWRKVKHAGRRVLDTAKGVGRHYLNNWLNRYRG

SEQ ID NO: 6 - protein, 30 aa, Myxine glutinosa
GWFKKAWRKVKNAGRVLKGVGIHYGVGLIG

SEQ ID NO: 7 - DNA, 498 bp, Capra hircus
  CDS (1)..(477)
atggagacccagagggccagcctctccctgggacggtgctccctgtggctcctgctgctgggactagtgctgccctcggccagcgcccaggccctcagctacggggaggccgttcttcatgctgtcgatcgcatcaatgagcagtcctcagaagcgaatctctaccgcctcctggagcttgacccgcctcccaaggacgatgagaatccaaacatcccgaaacctgtgagcttcagggtgaaggagactgtgtgccccaggacgagccggcagcccacggagcagtgtgacttcaaagagaatgggctggtgaagcaatgtgtagggacagtcactctggatgcggtgaaaggcaaaatgaacatcacctgcgaagagttgcagagtgttgggagatttaaacgatttcgtaagaagctcaaaagactctggcacaaagtcggcccattcgttggcccgatactccattatgggtaaattgtgagcccatggaagaat

SEQ ID NO: 8 - protein, 158 aa, Capra hircus
METQRASLSLGRCSLWLLLLGLVLPSASAQALSYGEAVLHAVDRINEQSSEANLYRLLELDPPPKDDENPNIPKPVSFRVKETVCPRTSRQPTEQCDFKENGLVKQCVGTVTLDAVKGKMNITCEELQSVGRFKRFRKKLKRLWHKVGPFVGPILHYG

SEQ ID NO: 9 - protein, 38 aa, Myxine glutinosa
  MISC_FEATURE (1)..(38): Xaa is Brominated Tryptophan
GFFKKAXRKVKHAGRRVLDTAKGVGRHYVNNXLNRYRZ

SEQ ID NO: 10 - protein, 31 aa, Myxine glutinosa
  MISC_FEATURE (1)..(31): Xaa is Brominated Tryptophan
GXFKKAXRKVKNAGRRVLKGVGIHYGVGLIZ

SEQ ID NO: 11 - DNA, 20 bp, Myxine glutinosa
  misc_feature (1)..(20): r = g or a
aaraargcntggmgnaargt

(19) United States
(12) Patent Application Publication
(10) Pub. No.: US 2004/0249143 A1
(43) Pub. Date: Dec. 9, 2004

(54) Hagfish cathelin-associated antimicrobial peptides and genes

(76) Inventors: Thomas Uzzell, Philadelphia, PA (US); Ann E. Shinnar, Plymouth Meeting, PA (US); Michael A. Zasloff, Plymouth Meeting, PA (US)

Correspondence Address: MORGAN LEWIS & BOCKIUS LLP, 1111 PENNSYLVANIA AVENUE NW, WASHINGTON, DC, 20004, US

(21) Appl. No.: 10/485,278

(22) PCT Filed: Jul. 30, 2002
(86) PCT No.: PCT/US02/24019

(30) Foreign Application Priority Data
Jul. 30, 2001 (US) 60308652

(51) Int. Cl. A61K 38/17; C07K 14/47; C07H 21/04

(52) U.S. Cl. 536/23.5; 530/324; 514/12; 435/69.1; 435/320.1; 435/325; 435/252.3

(57) ABSTRACT

The present invention includes <i>Myxine glutinosa</i> cathelin-associated antimicrobial peptides and genes encoding these peptides. The invention also includes compositions an methods for producing these peptides as well as method of preventing and treating microbial infections using these peptides.

### CROSS-REFERENCE TO RELATED APPLICATION

This application claims the benefit of U.S. provisional application No. 60/308,652 filed on Jul. 30, 2002.

### FIELD OF THE INVENTION

This invention relates to genes for antibiotic peptides and their use for the production of said peptides. It also relates to the antimicrobial peptides and their use as antibiotics.

### BACKGROUND OF THE INVENTION

The cathelicidin gene family (Zanetti et al. (1995) FEBS Letters 374, 1-5; Zanetti et al. (1997) Ann. New York. Acad. Sci. 147-162; Gennaro et al. (2000) Biopolymers Peptide Science 55, 3149) encodes prepropeptides that consist of a signal sequence, a cathelin-like acidic spacer, and an antibiotic peptide FIG. 1). Cathelicidins are presently known from a variety of mammals: Bos taurus (cattle), Capra hircus (goat), Cavia porcellus (guinea pig), Equus caballus (horse), Homo sapiens (human), Mus musculus (mouse), Oryctolagus cunniculus (rabbit), Ovis aries (sheep), and Sus scrofa (pig). Although the known antibiotic peptides are remarkably diverse, both within and between mammalian species, the associated signal sequences and cathelin-like acidic spacers, as revealed by sequence analysis of cDNA clones, are as remarkably conserved within and among species as the antibiotic peptides themselves are diverse.

Mammalian cathelicidins are synthesized primarily in myeloid cells, and are abundant in circulating neutrophils. The structure of some 20 cathelicidin genes is known and regularly involves four exons, the last of which encodes the antibiotic peptide along with a few C-terminal residues of the cathelin. Clusters of cathelicidin genes numbering eight, eleven, and more have been reported in Ovis, Bos and Sus, respectively (Gennaro et al. (2000) Biopolymers Peptide Science 55, 3149).

Two antibiotic peptides have been isolated (Kaumaya et al. (1996) Peptides: Chemistry and Biology, Mayflower Scientific, pages 189-191) from intestinal tissue of Myxine glutinosa (Atlantic hagfish), a craniate chordate taxon only distantly related to the Mammalia. These peptides are unique among antimicrobial peptides in containing one or two brominated tryptophans. Herein are described hagfish cDNA sequences encoding two new antibiotic peptides from the same family as those isolated earlier. These sequences identify the Myxine antibiotic peptides as members of the cathelicidin family.

### SUMMARY OF THE INVENTION

The invention includes an isolated nucleic acid molecule selected from the group consisting of an isolated nucleic acid molecule that encodes the amino acid sequence of a Myxine glutinosa cathelin-associated antimicrobial peptide comprising SEQ ID NO: 2 or 4; and an isolated nucleic acid molecule, which hybridizes to the complement of a nucleic acid molecule comprising a nucleotide sequence encoding a Myxine glutinosa cathelin-associated antimicrobial peptide comprising SEQ ID NO: 2 or 4 under stringent conditions. In some embodiments, the nucleic acid molecule comprises SEQ ID NO: 1 and 3; nucleotides 4 to 540 of SEQ ID NO: 1; nucleotides 427 to 540 of SEQ ID NO: 1; nucleotides 2 to 505 of SEQ ID NO: 3; or nucleotides 416 to 505 of SEQ ID NO: 3.

The invention also includes an isolated nucleic acid molecule selected from the group consisting of an isolated nucleic acid molecule that encodes the amino acid sequence of a Myxine glutinosa cathelin-associated antimicrobial peptide comprising SEQ ID NO: 5 or 6; and an isolated nucleic acid molecule, which hybridizes to the complement of a nucleic acid molecule comprising a nucleotide sequence encoding a Myxine glutinosa cathelin-associated antimicrobial peptide comprising SEQ ID NO: 5 or 6 under stringent conditions.

The isolated nucleic acid molecules of the invention can be operably linked to one or more expression control elements. The invention further includes a vector comprising an any of the isolated nucleic acid molecules recited above and a host cell comprising the vector.

The invention includes a method for producing a peptide or fragment thereof comprising the step of culturing the aforementioned host cell under conditions in which the protein or protein fragment encoded by said nucleic acid molecule is expressed. The host cell can be prokaryotic or eukaryotic. The invention includes recombinant peptides and fragments thereof produced by this method.

The invention includes an isolated peptide or fragment thereof selected from the group consisting of an isolated peptide comprising the amino acid sequences depicted in SEQ ID NO: 2, 4, 5 or 6; an isolated peptide fragment comprising at least six amino acids of any of the sequences depicted in SEQ ID NO: 2,4, 5 or 6; an isolated peptide comprising conservative amino acid substitutions of any of the sequences depicted in SEQ ID NO: 2, 4, 5 or 6; and naturally occurring amino acid sequence peptide variants of any of the amino acid sequences depicted in SEQ ID NO: 2,4, 5 or 6.

The invention further includes an antimicrobial composition comprising one or more of the peptides of of the invention. The antimicrobial composition can be suitable for topical or parenteral administration.

The invention includes a method of treating or preventing a microbial infection in a mammal, comprising administering to the mammal an effective amount of a Myxine glutinosa cathelin-associated antimicrobial peptide. In some embodiments, the Myxine glutinosa cathelin-associated antimicrobial peptide is selected from the group consisting of SEQ ID NO: 2, 4, 5 or 6 and the mammal is a human at risk of, or suffering from, a bacterial infection or a fungal infection. The Myxine glutinosa cathelin-associated antimicrobial peptide containing compositions can be administered by a route selected from the group consisting of oral, topical and parenteral. When topical administration is employed, it can be accomplished by inhalation. When parenteral administration is employed, it can be accomplished by intravenous, subcutaneous or intramuscular administration.

### BRIEF DESCRIPTION OF THE FIGURES

FIG. 1 depicts a comparison of nucleotide (SEQ ID NO: 1 and 3) and amino acid sequences (SEQ ID NO: 2 and 4) for hagfish cathelin-associated antimicrobial peptides. Beginnings of presumed signal, cathelin, peptide, and 3′ untranslated regions are marked. The peptide amino acids are italicized and in bold; start and stop codons are in upper case; the polyadenylation site is underlined. Dots indicate identical nucleotides of amino acids while dashes indicate insertions or deletions. * indicates the putative C-terminal amidation site in the antibiotic peptides.

FIG. 2 depicts a comparison of nucleotides and implied amino acid residues of SEQ ID NOS. 3 and 4 with Capra hircus MAP28. Borders between exons suggested by mammalian gene structures are marked by vertical bars.

FIG. 3 depicts a comparison of the amino acid sequences of the hagfish antibiotic peptides (SEQ ID NO: 5 and 6). Dashes indicate gaps inserted to maximize the alignment.

FIG. 4 depicts a comparison of amino acid sequences for hagfish cathelin-associated antimicrobial peptides (SEQ ID NO: 5 and 6) with antimicrobial peptides isolated from hagfish intestinal tissues (SEQ ID NO: 9 and 10) described in U.S. Pat. No. 5,734,015. Dashes indicate gaps inserted to maximize the alignment. X=Br-Trp and Z indicates a C-terminal amide.

### DETAILED DESCRIPTION

General

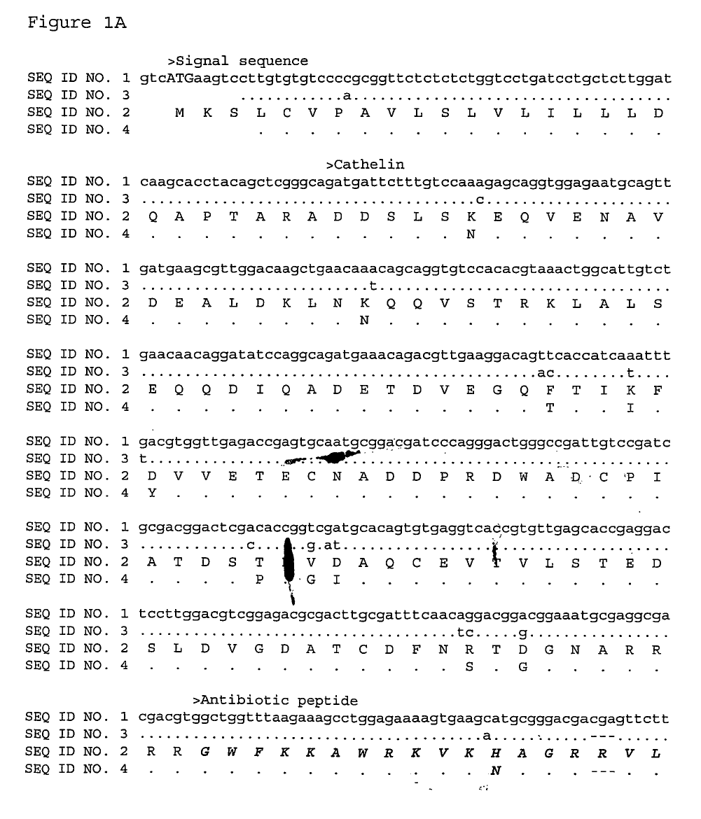

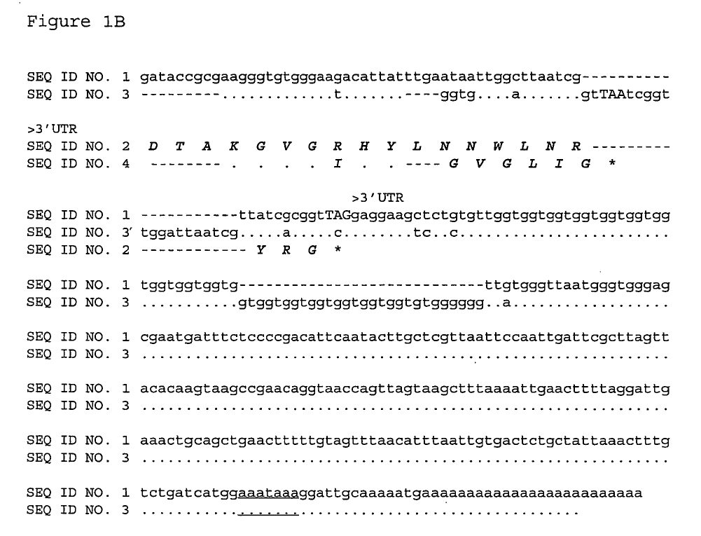

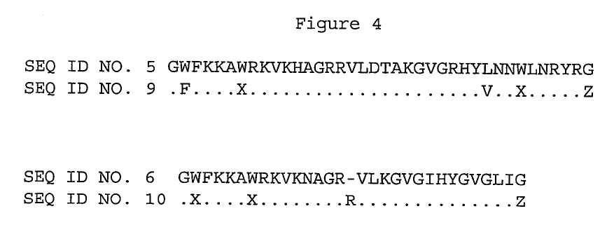

Applicants have isolated and sequenced two new genes from the Atlantic hagfish (Myxine glutinosa). The genes (SEQ ID NO: 1 and 3, FIG. 1) encode two proteins (SEQ ID NO: 2 and 4, FIG. 1) which each comprise an N-terminal signal sequence, a cathelin-like sequence and a C-terminal antimicrobial peptide. The antimicrobial peptide sequences deduced from these genes, GWFKKAWRKVKHAGRRVLDTAKGVGRHYLNNWLNRYRG (SEQ ID NO: 5) and GWFKKAWRKVKNAGRVLKGVGIHYGVGLIG (SEQ ID NO: 6), are highly similar yet distinct from the hagfish peptides described in U.S. Pat. No. 5,734,015 (FIG. 4). Thus, the invention includes the isolated hagfish genes and their encoded cathelin-associated antibiotic peptides.

Included in this invention is the antibiotic peptides wherein the C-terminal glycine residue has been post-translationally transformed into a C-terminal amide residue (SEQ ID NO: 11 and 12) by an enzyme such as peptidylglycine α-amidating monooxygenase (PAM) (Prigge et al. (2000) Cell. Mol. Life Sci. 57, 1236-1259) and/or one or more of the Trp residues have been post-translationally transformed into Br-Trp residues by a brominating enzyme (Shinnar et al. (2000) FASEB Journal 14, A1488).

Also included in the invention is the family of hagfish cathelin-associated genes, their encoded proteins and their antibiotic peptides. Another embodiment is methods for the expression of these genes and cells that contain them. The recombinant production of the proteins encoded by the hagfish cathelin-associated genes is included in this invention, as is the production of the associated antibiotic peptides.

Definitions

As used herein, the term “antimicrobial” refers to the ability of a compound to inhibit or irreversibly prevent the growth of a microorganism. Such inhibition or prevention can be through a microbicidal action or microbistatic inhibition. Therefore, the term “microbicidal inhibition” as used herein, refers to the ability of a compound to kill, or irreversibly damage the target organism. The term “microbistatic inhibition” as used herein, refers to the ability of the antimicrobial compound to inhibit the growth of the target microorganism without death. Microbicidal or microbistatic inhibition of microorganisms in an environment presently exhibiting microbial growth (therapeutic treatment) or an environment at risk of supporting microbial growth (prevention) is included within this definition.

As used herein, a “cell line” is a clone of a primary cell that is capable of stable growth in vitro for many generations.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A polyadenylation signal and transcription termination sequence will usually be located 3′ to the coding sequence.

As used herein, a “nucleic acid molecule” refers to the polymeric form of deoxyribonucleotides (adenine, guanine, thymine, and/or cytosine) or ribonucleotides (adenine, guanine, uracil, and/or cytosine) and may include in either its single stranded form, or in double-stranded helix as well as RNA. This term refers only to the primary and secondary structure of the molecule and is not limited to any particular tertiary form. In discussing the structure of particular double-stranded DNA molecules, sequences may be described herein according to the normal convention of giving only the sequence in the 5′ to 3′ direction along the nontranscribed strand of DNA (e.g., the strand having a sequence homologous to the mRNA). Transcriptional and translational control sequences are DNA regulatory sequences, such as promoters, enhancers, polyadenylation signals, terminators, and the like, that provide for the expression of a coding sequence in a host cell.

As used herein, a “promoter sequence” is a DNA regulatory region capable of binding RNA polymerase in a cell and initiating transcription of a downstream (3′ direction) coding sequence. For purposes of defining the present invention, the promoter sequence is bounded (inclusively) at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes.

A “signal sequence” can be included before the coding sequence or the native amino acid signal sequence from the envelope protein may be used. This sequence encodes a signal peptide, N-terminal to the polypeptide, that communicates to the host cell to direct the polypeptide to the cell surface or secrete the polypeptide into the media. This signal peptide is clipped off by the host cell before the protein leaves the cell. Signal sequences can be found associated with a variety of proteins native to prokaryotes and eukaryotes. For instance, alpha-factor, a native yeast protein, is secreted from yeast, and its signal sequence can be attached to heterologous proteins to be secreted into the media (see e.g., U.S. Pat. No. 4,546,082 and EP 0116201). Further, the alpha-factor and its analogs have been found to secrete heterologous proteins from a variety of yeast, such as Saccharomyces and Kluyveromyces (EP 88312306.9; EP 0324274 publication, and EP 0301669). An example for use in mammalian cells is the tPA signal used for expressing Factor VIIIc light chain.

A cell has been “transformed” by exogenous or heterologous DNA when such DNA as been introduced inside the cell. The transforming DNA may or may not be integrated (covalently linked) into chromosomal DNA making up the genome of the cell. In prokaryotes, for example, the transforming DNA may be maintained on an episomal element such as a plasmid or viral vector. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

A coding sequence is “under the control” of transcriptional and translational control sequences in a cell when RNA polymerase transcribes the coding sequence into mRNA, which is then translated into the protein encoded by the coding sequence.

As used herein, a “vector” is a replicon, such as plasmid, virus, phage or cosmid, to which another DNA segment may be attached so as to bring about the replication of the attached segment.

Nucleic Acid Molecules

The two cDNA sequences (FIG. 1) consist of 848 (SEQ ID NO: 1) and 863 (SEQ ID NO: 3) nucleotides. The sequence for SEQ ID NO: 1 includes the initiation codon and 3 additional 5′-nucleotides; the sequence for SEQ ID NO: 3, judged by alignment with SEQ ID NO: 1, has eight coding nucleotides missing at the 5′-end. In the alignment of FIG. 1, 814 nucleotides are comparable between the two sequences, with 785 matching (96.4%). The nucleotides encoding the putative signal and cathelin sequences of the two are very similar, with 69 of 70 signal nucleotides identical (98.6%) and 332 of 345 (96.2%) cathelin nucleotides matching; 401 of 415 (96.6%) match overall. Neither insertions nor deletions are evident in these parts of the cDNA sequences.

The peptide and 3′-untranslated sequences, in contrast, show evidence of several insertion/deletion changes, which makes comparison of their similarities difficult. In the peptide-encoding sequence, SEQ ID NO: 3 has three gaps (of 3, 9, and 4 nucleotides) in comparison to SEQ ID NO: 1; the last gap results in a frame-shift for 17 nucleotides just before the termination codon. The peptide-encoding sequence of SEQ ID NO: 1 has a gap of 21 nucleotides with respect to the sequence for SEQ ID NO: 3; as a result, a new termination codon 26 nucleotides farther along the sequence marks the end of the peptide for SEQ ID NO: 1. In this alignment, the last 10 coding nucleotides for SEQ ID NO: 1 correspond to 10 nucleotides in the 3′-untranslated region of the SEQ ID NO: 3 cDNA. Overall, 98 nucleotides encoding peptide can be compared; 89 of these (90.8%) match.

The 3 ′-untranslated ends of both cDNAs have -tgg- repeats, a 10-mer for SEQ ID NO: 1, and a 17-mer for SEQ ID NO: 3. Excluding the -tgg- repeats, 248 nucleotides in the 3′-end can be compared; of these, 243 (98.0%) match.

The nucleic acid molecules of the present invention include nucleic acid molecules that encode the proteins having SEQ ID NO: 2, SEQ ID NO: 4 and the related proteins herein described, preferably in isolated form. As used herein, “nucleic acid” is defined as RNA or DNA that encodes a protein or peptide as defined above, or is complementary to nucleic acid sequence encoding such peptides, or hybridizes to such nucleic acid and remains stably bound to it under appropriate stringency conditions, or encodes a polypeptide sharing at least 55% sequence identity, 85% sequence identity, preferably at least 85%, and more preferably at least 90%, most preferably at least 95 to 99% sequence identity with the peptide sequences. Specifically contemplated are genomic DNA, cDNA, mRNA and antisense molecules, as well as nucleic acids based on alternative backbones or including alternative bases whether derived from natural sources or synthesized. Such hybridizing or complementary nucleic acids, however, are defined further as being novel and unobvious over any prior art nucleic acid including that which encodes, hybridizes under appropriate stringency conditions, or is complementary to nucleic acid encoding a protein according to the present invention.

A preferred embodiment of the present invention is a nucleic acid molecule that encodes a hagfish cathelin-associated protein of the invention, such as a nucleic acid molecule comprising, consisting essentially of or consisting of SEQ ID NO: 1, or the open reading frame defined by nucleotides 4-540 (or 543) of SEQ ID NO: 1; or such as a nucleic acid molecule comprising, consisting essentially of or consisting of SEQ ID NO: 3, or the open reading frame defined by nucleotides 2-505 (or 508) of SEQ ID NO: 3. A further preferred embodiment of the present invention is a nucleic acid molecule that encodes a hagfish antimicrobial peptide of the invention, such as a nucleic acid molecule comprising, consisting essentially of or consisting of nucleotides 427-540 of SEQ ID NO: 1, encoding amino acids 142-179 of SEQ ID NO: 2, or encoding SEQ ID NO: 5; or such as a nucleic acid molecule comprising, consisting essentially of or consisting of nucleotides 416-505 of SEQ ID NO: 3, encoding amino acids 139-168 of SEQ ID NO: 4, or encoding SEQ ID NO: 6.

Homology or sequence identity at the nucleotide or amino acid sequence level is determined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference), recommended for query sequences over 85 nucleotides or amino acids in length.

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer (pH 6.5) with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal. Preferred nucleic acid molecules are those that hybridize under the above conditions to the complement of SEQ ID NO: 1 or 3 and which encode a polypeptide with antimicrobial activity. Even more preferred nucleic acid molecules are those that hybridize under the above conditions to the complement strand of the open reading frame of SEQ ID NO: 1 or 3.

As used herein, an “isolated” nucleic acid molecule(s) means a nucleic acid molecule, DNA or RNA, which has been removed from its native environment or when the nucleic acid molecule is substantially separated from contaminant nucleic acid encoding other polypeptides from the source of nucleic acid. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically. Isolated nucleic acid molecules of the present invention include DNA molecules comprising an open reading frame (ORF) shown in SEQ ID NO: 1 or 3; DNA molecules comprising the coding sequence for the mature antimicrobial peptide protein; and DNA molecules which comprise a sequence substantially different from those described above but which, due to the degeneracy of the genetic code, still encode the antimicrobial peptide protein. As the genetic code is well known in the art, it would be routine for one skilled in the art to generate such degenerate variants. In another aspect, the invention provides an isolated nucleic acid molecule comprising a polynucleotide which hybridizes under stringent hybridization conditions to a portion of the polynucleotide in a nucleic acid molecule of the invention. The present invention further relates to variants of the nucleic acid molecules of the present invention, which encode portions, analogs or derivatives of the antimicrobial peptide protein. Variants may occur naturally, such as a natural allelic variant. By an “allelic variant” is intended one of several alternate forms of a gene occupying a given locus on a chromosome of an organism.

The present invention further provides fragments of the encoding nucleic acid molecule. As used herein, a fragment of an encoding nucleic acid molecule refers to a small portion of the entire protein encoding sequence. The size of the fragment will be determined by the intended use. For example, if the fragment is chosen so as to encode an active portion of the protein, the fragment will need to be large enough to encode the functional region(s) of the protein or may encode regions of homology between the hagfish proteins in FIG. 1 (SEQ ID NO: 2 and 4). In one embodiment of the invention an antimicrobial peptide fragment will be preferably at least 15-20 amino acids, more preferably 20-30 amino acids and most preferably 30-40 amino acids. If the fragment is to be used as a nucleic acid probe or PCR primer, then the fragment length is chosen so as to obtain a relatively small number of false positives during probing/priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention can easily be synthesized by chemical techniques, for example, the phosphotriester method of Matteucci et al. (1981) J. Am. Chem. Soc. 103, 3185-3191 or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled encoding nucleic acid molecule.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

Isolation of Other Related Nucleic Acid Molecules

As described above, the identification of the hagfish nucleic acid molecules having SEQ ID NO: 1 and 3 allows a skilled artisan to isolate nucleic acid molecules that encode other members of the cathelin-associated protein family in addition to the sequences herein described.

Essentially, a skilled artisan can readily use the amino acid sequence of SEQ ID NO: 2 or 4 to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as a λgtll library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the protein.

Alternatively, a portion of the coding sequence herein described can be synthesized and used as a probe to retrieve DNA encoding a member of the hagfish cathelin-associated protein family from any mammalian organism. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

Recombinant Nucleic Acid Molecules

The present invention further provides recombinant DNA molecules (rDNA) that contain a hagfish cathelin-associated protein coding sequence. As used herein, an rDNA molecule is a DNA molecule that has been subjected to molecular manipulation in vitro. Methods for generating rDNA molecules are well known in the art (see, e.g., Sambrook et al. (2001) Molecular Cloning: A Laboratory Manual, Cold Spring Harbor Laboratory Press). In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extracbromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits RNA polymerase to bind and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 (Bio-Rad Laboratories), pPL and pKK223 (Pharmacia).

Expression vectors compatible with eukaryotic cells, preferably those compatible with vertebrate cells, can also be used to form an rDNA molecules that contains a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies), pTDT1 (ATCC), the vector pCDM8 described herein, and the like eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene (Southern, P.J. et al. 1982. J Mol Appl Genet 1(4): 327-41). Alternatively, the selectable marker can be present on a separate plasmid, and the two vectors are introduced by co-transfection of the host cell, and selected by culturing in the appropriate drug for the selectable marker.

Host Cells Containing an Exogenously Supplied Coding Nucleic Acid Molecule

The present invention further provides host cells transformed with a nucleic acid molecule that encodes a hagfish cathelin-associated protein, preferably SEQ ID NO: 1 or 3, of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect, and mammalian cells, preferably those from a mouse, rat, monkey or human cell line. Preferred eukaryotic host cells include cell lines established from hagfish cells, Chinese hamster ovary (CHO) cells available from the ATCC as CCL61, NIH Swiss mouse embryo cells NIH/3T3 available from the ATCC as CRL 1658, baby hamster kidney cells (BHK), and the like eukaryotic tissue culture cell lines.

Any prokaryotic host can be used to express an rDNA molecule encoding a protein of the invention. The preferred prokaryotic host is E. coli.

Transformation of appropriate cell hosts with an rDNA molecule of the present invention is accomplished by well known methods that typically depend on the type of vector used and host system employed. With regard to transformation of prokaryotic host cells, electroporation and salt treatment methods are typically employed (see, e.g., Cohen et al. (1972) Proc. Natl. Acad. Sci. USA 69, 2110-2112 and Sambrook et al. (2001) Molecular Cloning, A Laboratory Manual, Cold Spring Harbor Laboratory Press). With regard to transformation of vertebrate cells with vectors containing rDNAs, electroporation, cationic, lipid or salt treatment methods are typically employed (see, e.g., Graham et al. (1973) Virol. 52, 456-458; Wigler et al. (1979) Proc. Natl. Acad. Sci. USA 76, 1373-1376).

Successfully transformed cells (i.e., cells that contain an rDNA molecule of the present invention) can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern (1975) J. Mol. Biol. 98, 503-506) or Berent et al. (1985) Biotech. 3, 208-209) or the proteins produced from the cell assayed via an immunological method.

Production of Recombinant Proteins

The present invention further provides methods for producing a hagfish cathelin-associated protein of the invention using nucleic acid molecules herein described. In general terms, the production of a recombinant form of a protein typically involves the following steps:

First, a nucleic acid molecule is obtained that encodes a hagfish cathelin-associated protein of the invention, such as a nucleic acid molecule comprising, consisting essentially of or consisting of SEQ ID NO: 1, or the open reading frame defined by nucleotides 4-540 (or 543) of SEQ ID NO: 1; or such as a nucleic acid molecule comprising, consisting essentially of or consisting of SEQ ID NO: 3, or the open reading frame defined by nucleotides 2-505 (or 508) of SEQ ID NO: 3. Alternatively, a nucleic acid molecule is obtained that encodes a hagfish antimicrobial peptide of the invention, such as a nucleic acid molecule comprising, consisting essentially of or consisting of nucleotides 427-540 of SEQ ID NO: 1, encoding amino acid residues 142-179 of SEQ ID NO: 2, or encoding SEQ ID NO: 5; or such as a nucleic acid molecule comprising, consisting essentially of or consisting of nucleotides 416-505 of SEQ ID NO: 3, encoding amino acid residues 139-168 of SEQ ID NO: 4, or encoding SEQ ID NO: 6. If the encoding sequence is uninterrupted by introns, as is this open reading frame, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

In another embodiment, the polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

Hagfish Cathelin-associated Proteins

The amino acid sequences, SEQ ID NO: 2 and SEQ ID NO: 4 (FIG. 1) differ mainly because of differences in the region of the peptide associated with antimicrobial activity. The signal sequences are identical, with 26 residues; and the cathelins share 91.3% of their 115 residues. In contrast, the antimicrobial peptide consisting of amino acid residues 142-179 of SEQ ID NO: 2, GWFKKAWRKVKHAGRRVLDTAKGVGRHYLNNWLNRYRG (SEQ ID NO: 5), has 38 residues compared to 30 for the antimicrobial peptide consisting of amino acid residues 139-168 from SEQ ID NO: 4, GWFKKAWRKVKNAGRVLKGVGIHYGVGLIG (SEQ ID NO: 6). When three gaps are inserted in SEQ ID NO: 6, however, 23 of the 30 residues (76%) are identical to those of SEQ ID NO: 5. Both SEQ ID NO: 5 and SEQ ID NO: 6, have a C-terminal glycine residue which is likely post-translationally converted to a C-terminal amide as is the case with the hagfish antimicrobial peptides described in U.S. Pat. No. 5,734,015.

As used herein, the family of proteins related to the hagfish cathelin-associated proteins, refers to other cathelin-associated proteins that can be isolated from the hagfish or organisms closely related to the hagfish. The methods used to identify and isolate other members of the family of proteins related to the hagfish cathelin-associated proteins are described below.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain an isolated protein. By isolated, it is further meant that the hagfish cathelin-associated protein or the antimicrobial peptide of the present invention is part of a mixture with other components and wherein the protein or peptide of the present invention comprises at least about 10% of the total protein in the mixture, preferably at least about 20% of the total protein in the mixture, more preferably at least about 30% of the total protein in the mixture, even more preferably at least about 40% of the total protein in the mixture, still more preferably at least about 50% of the total protein in the mixture, yet more preferably at least about 60% of the total protein in the mixture, even still more preferably at least about 70% of the total protein in the mixture, yet still more preferably at least about 80% of the total protein in the mixture, much more preferably at least about 90% of the total protein in the mixture, still much more preferably at least about 95% of the total protein in the mixture, and most preferably at least about 99% of the total protein in the mixture.

The proteins of the present invention further include conservative variants of the proteins herein described. As used herein, a conservative variant refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein may be altered without adversely affecting a biological activity. Accordingly, the amino acid sequence can be altered, for example to render the peptide more hydrophobic or hydrophilic, without adversely affecting the biological activities of the protein.

Ordinarily, the allelic variants, the conservative substitution variants, and the members of the protein family, will have an amino acid sequence having at least about 85% amino acid sequence identity with the sequences set forth in SEQ ID NO: 2, 4, 5 or 6, more preferably at least 90%, even more preferably at least 95% and most preferably at least 99%. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N-terminal, C-terminal or internal extensions, deletions, or insertions into the peptide sequence shall not be construed as affecting homology.

Thus, the proteins of the present invention include molecules comprising, consisting essentially of, or consisting of the amino acid sequence disclosed in SEQ ID NO: 2, 4, 5 and 6; fragments thereof having a consecutive sequence of at least about 3, 4, 5, 6, 10, 15, 20, 25, 30, 35, 38 or more amino acid residues of the hagfish cathelin-associated protein or antimicrobial peptide thereof; amino acid sequence variants of such sequence wherein an amino acid residue has been inserted N- or C-terminal to, or within, the disclosed sequence; and amino acid sequence variants of the disclosed sequence, or their fragments as defined above, that have been substituted by another residue. Contemplated variants further include those containing predetermined mutations by, e.g., homologous recombination, site-directed or PCR mutagenesis, and the corresponding proteins of other animal species, including but not limited to rabbit, rat, porcine, bovine, ovine, equine and non-human primate species, and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

Therapeutic and Additive Applications

Another aspect of the present invention is a method for inhibiting microbial growth. Hagfish cathelin-associated antimicrobial peptides of the present invention, exemplified by SEQ ID NOs: 5 and 6, can be used to inhibit microbial growth under various circumstances. For example, the peptides of the present invention can be administered therapeutically to treat or prevent disease in an individual resulting from a microbial infection. A variety of microbial infections can be inhibited by treatment with the peptides of the present invention. For example, the antimicrobial peptides of the present invention can be used as an antimicrobial agent for the treatment of fungal or bacterial infections.

In preferred embodiments, the microbial infection is a bacterial infection. In particular, therapeutic administration of the peptides of the present invention inhibits and can reverse the progression of a bacterial infection. The development and spread of new resistant strains of bacteria are increasingly posing a public health threat. The antimicrobial peptides of the present invention are useful in treating patients suffering from these resistant strains of microbes which currently threaten public health. Many bacterial infections cause bacterial sepsis in the infected individual. Administration of the peptides to an individual suffering from a septic bacterial infection produces a significant therapeutic effect, by reducing both the sepsis caused by the infection, and the infection itself.

An individual suitable for treatment is any animal (mammal or otherwise) which is afflicted with or otherwise susceptible to one or more of the above described microbial infections. In a preferred embodiment, the individual is a human. In another embodiment, the individual is a livestock animal. In another embodiment, the animal is a show animal or a household pet.

Administration of the hagfish cathelin-associated antimicrobial peptides of the present invention to the individual is either systemic or localized, and is largely determined by the specific infection being treated. Systemic administration can be accomplished by several routes, including but not limited to intravenous administration, inhalation, mucosal, and ingestion. Localized administration can be topical or internal. Such administration can be accomplished by several routes, including, but not limited to, subcutaneous, dermal, intradermal, buccal, mucosal, intraperitoneal, vaginal, inhalation, and ingestion.

It will often be of use to administer a formulation of the peptides of the present invention which includes a pharmaceutically acceptable carrier. Possible formulations for therapeutic administration include a variety of pharmaceutical compositions, the appropriate use of which will depend upon the route of administration deemed necessary for treatment. Some useful formulations for topical administration are, for example, eyedrops, eardrops, or gingival applications (e.g., drops, mouthwash, cream, or paste). The regimen of administration (e.g., route, dose, and course) which is therapeutic to the patient will vary with the individual to be treated (e.g., health, weight, metabolism), the site of infection, and the infecting pathogen. A therapeutic regimen should be developed by extrapolation from treatment with similar therapeutics in combination with empirical observation. Administration of the peptides of the present invention to prevent a microbial infection in an individual parallel the above described methods.

As a general proposition, the total pharmaceutically effective amount of antimicrobial peptide administered parenterally per dose will be in the range of about 0.001 mg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the peptide. If given continuously, the antimicrobial peptide is typically administered at a dose rate of about 0.001 mg/kg/hour to about 0.050 mg/kg/hour, either by one to four injections per day or by continuous subcutaneous infusions, for example, using a mini-pump. An intravenous bag solution may also be employed.

Hagfish cathelin-associated antimicrobial peptides of the present invention, exemplified by SEQ ID NO: 5 and 6, are also useful for potentiating the therapeutic action of other antimicrobial drugs or agents. Co-administration of other antimicrobial agents with the antimicrobial polypeptides of the present invention, produce a synergistic antibiotic effect. Co-administration of the peptides of the present invention with an antimicrobial agent enables therapeutic treatment of a patient with lower doses of the antimicrobial agent. Lower doses are preferable in situations such as when treating with an expensive drug, or one that produces undesired side effects, or one whose short half-life in vivo would otherwise rapidly reduce its concentration below that which is required for it to be efficacious. In addition, co-administration with antimicrobial agents or drugs may also allow for a shorter therapy period and/or the reversal of resistant phenotypes. Without limitation, microbes which resist an antimicrobial drug by decreasing their internal drug concentration (e.g., with decreased membrane permeability or increased cellular export or metabolism of the drug) are expected to be especially susceptible to the potentiating activity of these peptides.

The regimen of administration of the antimicrobial drug and the hagfish cathelin-associated antimicrobial peptide or functional variant of the present invention varies with the patient and the particular infection, and can be determined by one of skill in the art on a case by case basis. Formulations of the polypeptide will depend upon the regimen of administration, examples described above.

The polypeptides of the present invention can be used not only as pharmaceutical and neutraceutical agents but also as additives for any products such as foods and medicinal or non-medicinal products which are taken into the bodies or otherwise applied onto or contacted with the body surface of humans or other animals or fluids, organs, and cells derived therefrom.

The present method is useful for treating a variety of products. Biological products, defined herein as products which are derived from biological organisms or processes, are particularly at risk for contamination with microorganisms. Examples of biological products include without limitation, food products, tissue, living cells, products derived from living cells, blood or components thereof, as any other bodily fluid, drugs or other molecular preparations. Non-biological products, defined herein as a product not directly derived from a biological organism or process, for example glassware, surgical equipment, synthetic drugs or other molecular preparations, can also be treated. For effective use of the present invention, the product which is to be treated should not possess an activity which completely inactivates the antimicrobial activity of all of the peptide quantity so applied. The peptides of the present invention can be added, assorted to, sprayed to, adhered to, coated onto, adsorbed to, chemically crosslinked to, or impregnated into any products which are generally desired to be prevented or inhibited from contamination by proliferating microorganisms. Alternatively, the peptide of the present invention can be immobilized on a surface over which a product is passed to remove or inhibit microbial growth in the product. A product which is treated with and retains antimicrobial peptide can further be used to treat another product with which it is contacted.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLE 1

Isolation of Genes for Hagfish Cathelin-associated Antibiotic Peptides

Adult Atlantic hagfish (Myxine glutinosa Linnaeus, 1758; Myxinidae) were purchased from Huntsman Marine Science Centre (New Brunswick, Canada). The fish were anesthetized in a slurry of ice and decapitated. Intestines were removed and cleaned of contents, frozen in liquid N₂, and stored at −80° C.

Tissue was ground in liquid nitrogen and transferred to a guanidinium isothiocyanate solution (Chirgwin et al. (1979) Biochemistry 18, 5294-5299), which inhibits RNase activity while disrupting cells. Total RNA was isolated by centrifugation through a CsCl step gradient; polyadenylated RNA was recovered by means of oligo(dT) cellulose (Stratagene). Reverse transcribed cDNA was ligated first to adaptor arms and then into phage Lambda Zap II following Stratagene protocols.

An twenty-mer oligonucleotide probe 5′-AARAARGCNTGGMGNAARGT-3′ (SEQ ID NO: 11; 256-fold degenerate) was designed based on amino acid residues 4 to 10 of the peptides previously isolated from hagfish intestinal tissue. Phage particles containing homologous sequences were isolated by plating a small number of particles on a lawn of XL1 Blue MRF (Stratagene). The cDNA inserts were isolated in phagmid pBluescript by in vivo excision. Two inserts were sequenced in both directions using Sanger's dideoxy method (Barnes et al. (1983) Nucleic Acids Res. 11, 349-368).

Homologous sequences were identified using BLASIX (Altschul et al. (1997) Nucleic Acids Res. 25, 3389-3402) searches of GenBank. Homologous sequences were aligned using ESEE (Cabot et al. (1989) Compu. Appl. Biosci. 5, 233-234).

For in situ hybridizations, plasmid DNA containing insert 31-52 from SEQ D NO: 1 was linearized with StyI. Both sense and antisense probes were labeled enzymatically with ³²p and detected by autoradiography.

### EXAMPLE 2

Comparison of Hagfish Cathelin-associated Sequences with Other Cathelin-associated Sequences

Searches of GenBank using BLASTN with default values yielded no matches either when just the coding region for the two peptides were used as queries or when the entire coding regions were used. Using the entire coding sequence available for each protein and BLASIX, which searches for more distant relationships based on identical and conserved amino acids, however, yielded a large number of matches. Those in the correct reading frame included especially cDNA sequences for prepropeptides of mammalian antibiotic peptides containing a cathelin-like acidic spacer.

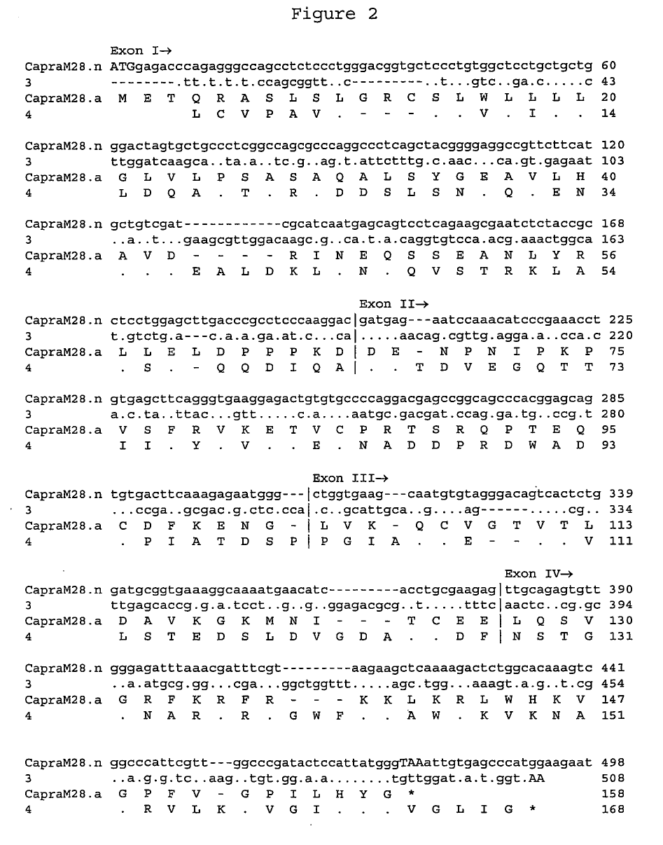

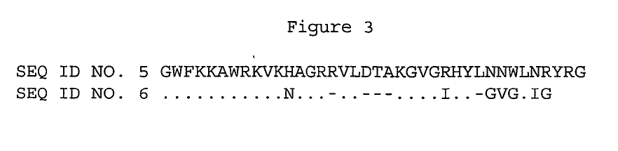

Among the matches found, the sequence for Capra MAP28, isolated from bone marrow cells of Capra hircus (goat) was unique in showing at least a weak match between the antibiotic peptide and one of the two Myxine peptides. A plausible alignment of the coding regions of one of the hagfish sequences with Capra MAP28 is shown in FIG. 2.

### EXAMPLE 3

Antimicrobial Activity of Cathelin-associated Antibiotic Peptides

The antimicrobial activity of cathelin-associated antibiotic peptides is determined by the following antibacterial assay based upon the guidelines of the National Committee for Clinical Laboratory Standards (Document M7-T2, (1988) Volume 8, No. 8).

A stock solution of the test peptide is prepared at a concentration of 512 μg/ml in sterile deionized distilled water and stored at −70° C. The stock peptide solutions are diluted in serial dilutions (1:2) down the wells of a microtiter plate so that the final concentrations of peptide in the well are 0.25, 0.50, 1, 2, 4, 8, 16, 32, 64, 128 and 256 μg/ml. 1.5×10⁵ CFU/ml of the test microbe is added to the wells in full strength Mueller Hinton broth (BBL 11443) from a mid-log culture. The inoculum is standardized spectrophotometrically at 600 nm and is verified by colony counts. The plates are incubated for 16-24 hours at 37° C. and the minimal inhibitory concentration (MIC) for each peptide is determined. Minimal inhibitory concentration is defined as the lowest concentration of peptide which produces a clear well in the microtiter plate.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All publications, patents and patent applications referred to in this application are herein incorporated by reference in their entirety.

1. An isolated nucleic acid molecule selected from the group consisting of:
(a) an isolated nucleic acid molecule that encodes the amino acid sequence of a Myxine glutinosa cathelin-associated antimicrobial peptide comprising SEQ ID NO: 2 or 4; and
(b) an isolated nucleic acid molecule, which hybridizes to the complement of a nucleic acid molecule comprising a nucleotide sequence encoding a Myxine glutinosa cathelin-associated antimicrobial peptide comprising SEQ ID NO: 2 or 4 under stringent conditions.

2. The isolated nucleic acid molecule of claim 1, wherein the nucleic acid molecule is selected from the group consisting of SEQ ID NO: 1 and 3.

3. The isolated nucleic acid molecule of claim 2, wherein the nucleic acid molecule comprises nucleotides 4 to 540 of SEQ ID NO: 1.

4. The isolated nucleic acid molecule of claim 2, wherein the nucleic acid molecule comprises nucleotides 427 to 540 of SEQ ID NO: 1.

5. The isolated nucleic acid molecule of claim 1, wherein the nucleic acid molecule comprises nucleotides 2 to 505 of SEQ ID NO: 3.

6. The isolated nucleic acid molecule of claim 1, wherein the nucleic acid molecule comprises nucleotides 416 to 505 of SEQ ID NO: 3.

7. An isolated nucleic acid molecule selected from the group consisting of:
(a) an isolated nucleic acid molecule that encodes the amino acid sequence of a Myxine glutinosa cathelin-associated antimicrobial peptide comprising SEQ ID NO: 5 or 6; and
(b) an isolated nucleic acid molecule, which hybridizes to the complement of a nucleic acid molecule comprising a nucleotide sequence encoding a Myxine glutinosa cathelin-associated antimicrobial peptide comprising SEQ ID NO: 5 or 6 under stringent conditions.

8. The isolated nucleic acid molecule of claim 1, wherein said nucleic acid molecule is operably linked to one or more expression control elements.

9. A vector comprising an isolated nucleic acid molecule of claim 1.

10. A host cell comprising a vector of claim 9.

11. A host cell transformed to contain the nucleic acid molecule of claim 1.

12. A method for producing a peptide or fragment thereof comprising the step of culturing a host cell of claim 11 under conditions in which the protein or protein fragment encoded by said nucleic acid molecule is expressed.

13. The method of claim 12, wherein said host cell is selected from the group consisting of prokaryotic hosts and eukaryotic hosts.

14. An isolated peptide or fragment thereof produced by the method of claim 12.

15. An isolated peptide or fragment thereof selected from the group consisting of:
(a) an isolated peptide comprising the amino acid sequences depicted in SEQ ID NO: 2, 4; 5 or 6;
(b) an isolated peptide fragment comprising at least six amino acids of any of the sequences depicted in SEQ ID NO: 2, 4, 5 or 6;
(c) an isolated peptide comprising conservative amino acid substitutions of any of the sequences depicted in SEQ ID NO: 2, 4, 5 or 6; and
(d) naturally occurring amino acid sequence peptide variants of any of the amino acid sequences depicted in SEQ ID NO: 2, 4, 5 or 6.

16. The isolated protein fragment of claim 15 wherein the isolated peptide consists essentially of the amino acid sequences depicted in SEQ ID NO: 2, 4, 5 or 6.

17. A antimicrobial composition comprising one or more of the peptides of claim 15.

18. The antimicrobial composition of claim 17, wherein the composition is suitable for topical or parenteral administration.

19. A method of treating or preventing a microbial infection in a mammal, comprising administering to the mammal an effective amount of a Myxine glutinosa cathelin-associated antimicrobial peptide.

20. The method of claim 19, wherein the Myxine glutinosa cathelin-associated antimicrobial peptide is selected from the group consisting of SEQ ID NO: 2, 4; 5 or 6.

21. The method of claim 19, wherein the mammal is a human.

22. The method of claim 19, wherein the microbial infection is selected from the group consisting of a bacterial infection and a fungal infection.

23. The method of claim 17, wherein the Myxine glutinosa cathelin-associated antimicrobial peptide is administered by a route selected from the group consisting of oral, topical and parenteral.

24. The method of claim 23, wherein the topical administration is accomplished by inhalation.

25. The method of claim 24, wherein the parenteral administration is accomplished by intravenous, subcutaneous or intramuscular administration.
